(19) United States
(12) Patent Application Publication
(10) Pub. No.: US 2017/0215029 A1
(43) Pub. Date: Jul. 27, 2017

(54) BIOSENSOR DEVICE, MANAGEMENT DEVICE, AND COMMUNICATION SYSTEM USED BY WORKERS UNDERTAKING DANGEROUS OPERATIONS

(71) Applicant: JVC KENWOOD Corporation, Yokohama-shi (JP)

(72) Inventors: Hideki MAKINOUCHI, Yokohama-shi (JP); Eiiti HOSONO, Yokohama-shi (JP)

(73) Assignee: JVC KENWOOD Corporation, Yokohama-shi (JP)

(21) Appl. No.: 15/408,456

(22) Filed: Jan. 18, 2017

(30) Foreign Application Priority Data
Jan. 26, 2016 (JP) 2016-012391

(51) Int. Cl. H04W 4/00; A61B 5/00; A61B 5/0205; H04W 36/24

(52) U.S. Cl. CPC H04W 4/008; H04W 36/24; A61B 5/002; A61B 5/02055; H04W 88/04

(57) ABSTRACT

A biological information acquisition unit acquires biological information on a subject person. A communication unit transmits the biological information acquired by the biological information acquisition unit to a terminal device that serves as a relay device for relaying the biological information to a management device. A connection destination determination unit, when changing a destination of connection from a currently connected terminal device to another terminal device, determines a terminal device as a new destination of connection in accordance with a content of the biological information relayed by the other terminal devices.

### CROSS-REFERENCE TO RELATED APPLICATION

This application is based upon and claims the benefit of priority from the prior Japanese Patent Application No. 2016-012391, filed on Jan. 26, 2016, the entire contents of which are incorporated herein by reference.

### BACKGROUND

1. Field of the Invention

The present invention relates to biosensor devices, management devices, and communication systems used by workers who undertake dangerous operations such as firefighting operations.

2. Description of the Related Art

A large number of firefighters die in the line of duty from heart attack or injury. To address the issue, a system is proposed in which a biosensor is worn by the body of a firefighter and biological information on the firefighter is transmitted wirelessly to a command center to monitor the safety of the firefighter on a real-time basis. For example, a firefighter is determined to be in a dangerous state if the heart rate of the firefighter is abnormal.

Patent Document 1 discloses a biological information management system comprised of a wearable device and a server. If any wearable device other than the wearer's device is located in the neighborhood of the wearer's device, a server connection device for connection to a server is determined adaptively based on the processing power and battery level of the wearer's device, the processing power and battery level of the wearable devices in the neighborhood, and conditions for communication. Biological data detected by a biosensor of the wearer's device is transmitted to the server via the server connection device.

Patent Document 2 discloses a biological information transmission device. The biological information transmission device transmits alert information by using a first wireless communication channel (e.g., Bluetooth (registered trademark)) and a second wireless communication channel (e.g., Wi-Fi (registered trademark)) when an abnormality is found in the biological information.
[patent document 1] JP2014-213071
[patent document 2] JP5237509

In the aforementioned system in which a biosensor is worn by the body of a firefighter and biological information on the firefighter is transmitted wirelessly to a command center, the battery of a wireless device may run out if the firefighting activity lasts for a long period of time. Another possibility is that the firefighter may move to a place not reached by radio waves during a firefighting activity. In either case, the wireless device of the firefighter cannot transmit the biological information on the firefighter to the command center.

### SUMMARY

To address the aforementioned issue, the biosensor device according to an embodiment comprises: a biological information acquisition unit that acquires biological information on a subject person; a communication unit that transmits the biological information acquired by the biological information acquisition unit to a communication terminal device that serves as a relay device for relaying the biological information to a management device; and a connection destination determination unit that, when changing a destination of connection from a currently connected communication terminal device to another communication terminal device, determines a communication terminal device as a new destination of connection in accordance with a content of the biological information relayed by the other communication terminal devices.

Another embodiment of the present invention relates to a management device. The device comprises: a communication unit that receives a signal including biological information on a subject person detected by a biosensor device, via a communication terminal device; and a connection destination determination unit that, when changing a relay device for a given biosensor device from a currently connected communication terminal device to another communication terminal device, determines a communication terminal device as a new destination of connection in accordance with a content of the biological information relayed by the other communication terminal devices.

Another embodiment of the present invention relates to a communication system. The system comprises: a biosensor device that acquires biological information on a subject person; a plurality of communication terminal devices that relay the biological information acquired by the biosensor device to a management device; and a management device that acquires the biological information on a plurality of persons from the plurality of communication terminals, wherein the management device: when changing a relay device for a given biosensor device from a currently connected communication terminal device to another communication terminal device, determines a communication terminal device as a new destination of connection in accordance with a content of the biological information relayed by the other communication terminal devices.

Another embodiment of the present invention relates to a communication method. The method comprises: using a biosensor device to acquire biological information on a subject person; using a communication terminal device to relay the biological information acquired to a management device; using the biosensor device to, when changing a destination of connection from a currently connected communication terminal device to another communication terminal device, determines a communication terminal device as a new destination of connection in accordance with a content of the biological information relayed by the other communication terminal devices.

Optional combinations of the aforementioned constituting elements, and implementations of the invention in the form of methods, apparatuses, systems, recording mediums, and computer programs may also be practiced as additional modes of the present invention.

### BRIEF DESCRIPTION OF THE DRAWINGS

Embodiments will now be described by way of examples only, with reference to the accompanying drawings which are meant to be exemplary, not limiting and wherein like elements are numbered alike in several Figures in which:

FIG. 1 shows the configuration of a dangerous operation support system according to the embodiments of the present invention;

FIG. 2 shows the configuration of the biosensor device according to Embodiment 1;

FIG. 3 shows the configuration of the terminal device according to Embodiment 1;

FIGS. 4A, 4B and 4C show the configuration of the management device and exemplary device information according to Embodiment 1;

FIGS. 5A and 5B is a flowchart showing the flow of the process in the dangerous operation support system 1 according to Embodiment 1;

FIGS. 6A and 6B show a specific example of switching destinations of pairing connection according to Embodiment 1 (Ex. 1);

FIGS. 7A and 7B show a specific example of switching destinations of pairing connection according to Embodiment 1 (Ex. 2);

FIG. 8 shows the configuration of the biosensor device according to Embodiment 2;

FIG. 9 shows the configuration of the terminal device according to Embodiment 2;

FIG. 10 shows the configuration of the management device according to Embodiment 2; and

FIGS. 11A and 11B is a flowchart showing the flow of the process in the dangerous operation support system according to Embodiment 2.

### DETAILED DESCRIPTION

The invention will now be described by reference to the preferred embodiments. This does not intend to limit the scope of the present invention, but to exemplify the invention.

Embodiments of the present invention relate to a dangerous operation support system for supporting firefighting operations of a firefighting team formed by multiple firefighters. In the following description, a firefighting operation is discussed as an example. A firefighting operation is given as an example of dangerous operations and the inventive system is applicable to dangerous operations in general in which multiple workers cooperate. For example, the system is applicable to rescue operations or recovery operations undertaken in the event of a natural disaster such as an earthquake or landslide. The system is also applicable to operations in dangerous places such as sites of mine development.

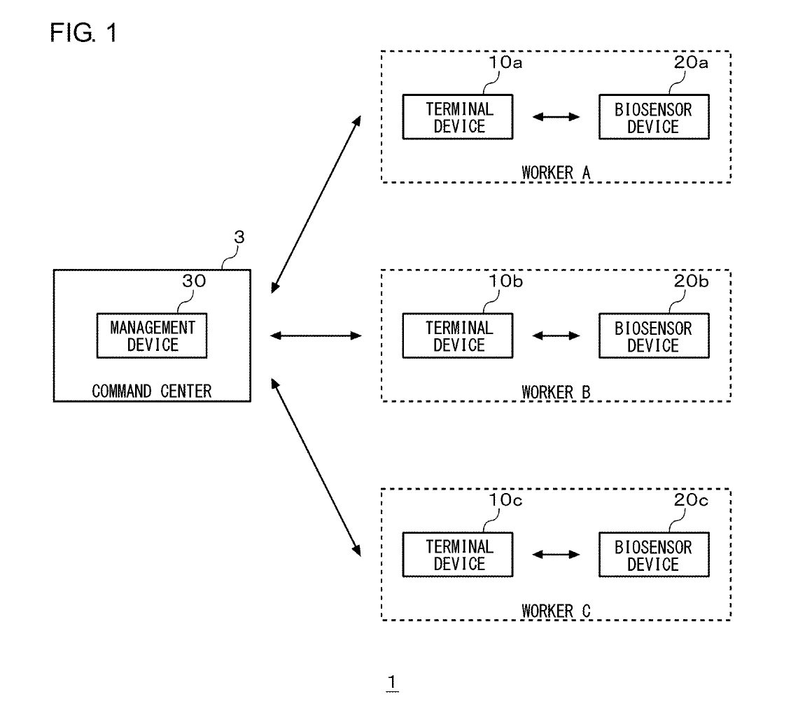

FIG. 1 shows the configuration of a dangerous operation support system 1 according to the embodiments of the present invention. The dangerous operation support system 1 includes a management device 30 installed at a command center 3, a biosensor device 20 worn by each worker, and a terminal device 10 held or carried by each worker. In this specification, a case of three workers is taken as an example.

The biosensor device 20 is worn by the body of the worker and detects biological information such as heart rate, body temperature, and posture. The biosensor device 20 is available in various types including a type glued to the body by a gel pad, a type wound around the arm or leg by a band, a type attached inside a clothing. Near field communication is established between the biosensor device 20 and the terminal device 10. In this specification, an example in which Bluetooth (registered trademark) is used as near field communication is described. Near field communication other than Bluetooth (registered trademark), such as Wi-Fi (registered trademark) and infrared communication, may be used for near field communication.

The terminal device 10 may be implemented by a communication terminal device such as a smartphone. In the description below, it is assumed that a smartphone is used. The management device 30 is installed at a place such as a fire station or command van that serves as the command center 3. The management device 30 may be implemented by a PC connected to an IP network. The terminal device 10 and the management device 30 are connected wirelessly by using a cell phone network. For example, the terminal device 10 accesses a base station device by using a Long-Term Evolution (LTE) cell phone network and communicates with the management device 30 via the base station device. The terminal device 10 may access an access point by using Wi-Fi (registered trademark) and communicate with the management device 30 via the access point.

Instead of a smartphone, a simplified mobile wireless device for business wireless communication may be used as the terminal device 10. In this case, the management device 30 is implemented by a PC connected to a base station wireless device for business wireless communication. The terminal device 10 and the management device 30 are connected by a business wireless communication system.

### Embodiment 1

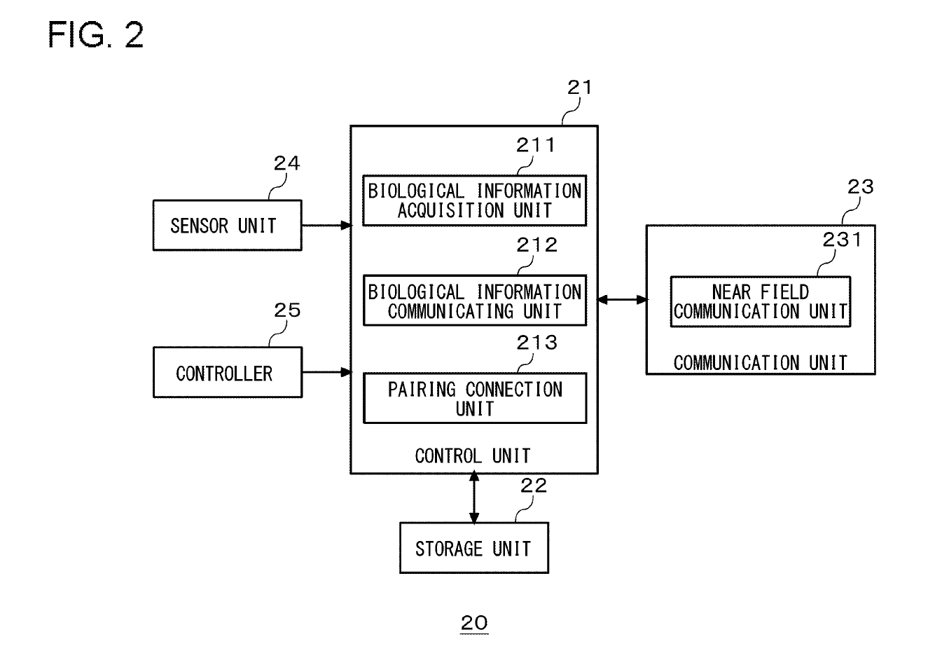

FIG. 2 shows the configuration of the biosensor device 20 according to Embodiment 1. The biosensor device 20 includes a control unit 21, a storage unit 22, a communication unit 23, a sensor unit 24, and a controller 25. The control unit 21 includes a biological information acquisition unit 211, a biological information communicating unit 212, and a pairing connection unit 213. The functions of the control unit 21 are implemented by the coordination of hardware resources and software resources, or hardware resources alone. A processor, ROM, RAM, and other LSIs can be used as hardware resources. Programs such as firmware can be used as software resources.

Identification information (hereinafter, also referred to as ID) uniquely identifying the biosensor device 20 is assigned to the biosensor device 20.

The storage unit 22 includes a storage medium such as a nonvolatile semiconductor memory. The communication unit 23 includes a near field communication unit 231. Medium or long distance wireless communication capabilities of a cell phone network, a business wireless communication system, etc. are not provided. The sensor unit 24 includes a heart rate sensor, a body temperature sensor, a three-axis acceleration sensor or the like to detect the heart rate, body temperature, posture or the like of the worker wearing the device. The sensor unit 24 outputs the detected biological information to the control unit 21.

The biological information acquisition unit 211 acquires the detected biological information from the sensor unit 24. The biological information communicating unit 212 periodically (e.g., at the intervals of several seconds) communicates the acquired biological information and the ID of the biosensor device 20 to the terminal device 10 paired with the biosensor device 20, via the near field communication unit 231.

The pairing connection unit 213 manages pairing connection in near field communication. More specifically, the pairing connection unit 213 discloses the existence of the host device to nearby devices provided with near field wireless communication functions according to an instruction from the controller 25. When the pairing connection unit 213 receives a request for connection from a device that functions as a master device in relation to the host device (slave device), the pairing connection unit 213 establishes a pairing connection with the requesting device according to a predetermined procedure. In this specification, it is assumed that a pairing connection is established with the terminal device 10 and the terminal device 10 functions as a master device and the biosensor device 20 functions as a slave device.

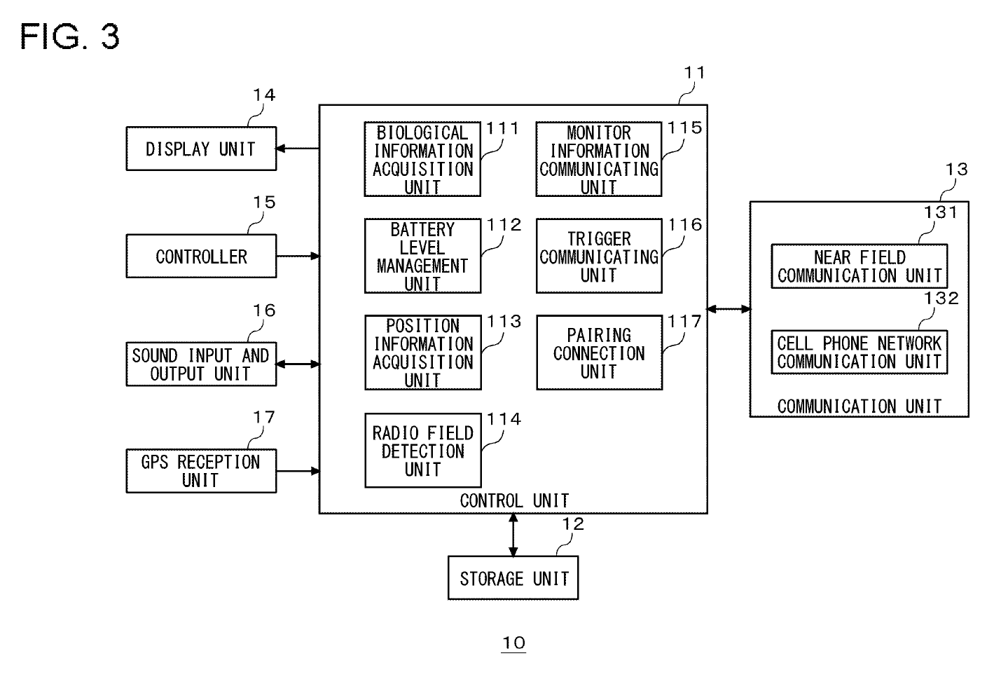

FIG. 3 shows the configuration of the terminal device 10 according to Embodiment 1. The terminal device 10 includes a control unit 11, a storage unit 12, a communication unit 13, a display unit 14, a controller 15, a sound input and output unit 16, and a GPS reception unit 17. The control unit 11 includes a biological information acquisition unit 111, a battery level management unit 112, a position information acquisition unit 113, an electric field detection unit 114, a monitor information communicating unit 115, a trigger communicating unit 116, and a pairing connection unit 117. The functions of the control unit 11 are also implemented by the coordination of hardware resources and software resources, or hardware resources alone. FIG. 3 depicts only those function blocks of the control unit 11 related to the process of interest in Embodiment 1.

Identification information (hereinafter, also referred to as ID) uniquely identifying the terminal device 10 is assigned to the terminal device 10.

The storage unit 12 includes a storage medium such as a nonvolatile semiconductor memory. The sound input and output unit 16 includes a microphone and a speaker. The GPS reception unit 17 receives signals from a plurality of GPS satellites and detects current location information based on the plurality of received signals. The position information is identified by latitude and longitude.

The communication unit 13 includes a near field communication unit 131 and a cell phone network communication unit 132. The near field communication unit 131 is used for communication with the biosensor device 20 and the cell phone network communication unit 132 is used for communication with the management device 30 or another terminal device 10.

The biological information acquisition unit 111 acquires the biological information on the firefighter (subject person) transmitted from the biosensor device 20 by near field communication and also acquires the ID of the biosensor device 20. The battery level management unit 112 manages the battery level of the terminal device 10. The position information acquisition unit 113 acquires the position information detected by the GPS reception unit 17. The electric field detection unit 114 detects the intensity of a signal transmitted from a base station device or an access point (not shown). In other words, the electric field detection unit 114 detects a condition of radio waves on the wireless communication channel used for communication with the management device 30.

The monitor information communicating unit 115 communicates monitor information including the ID of the terminal device 10, the biological information and the ID of the biosensor device 20 acquired by the biological information acquisition unit 111, the battery level managed by the battery level management unit 112, and the position information acquired by the position information acquisition unit 113 to the management device 30 via the cell phone network.

When a predetermined condition is met, the trigger communicating unit 116 communicates a trigger signal to the management device 30. For example, a trigger signal may be communicated when the battery level of the terminal device 10 is below a preset value (e.g., 5%) or when a trigger button in the controller 15 is pressed. Alternatively, a trigger signal may be communicated when the radio field intensity detected by the electric field detection unit 114 is below a preset value (e.g., −110 db).

The pairing connection unit 117 manages pairing connection in near field communication. More specifically, when a search instruction to search for nearby devices provided with near field wireless communication functions is entered in the controller 15, the pairing connection unit 117 searches for devices provided with near field wireless communication functions. In response to the search, devices located in a range reached by radio waves of near field communication and disclosing their existence are detected as candidates of pairing connection. When one of the candidates is selected according to an instruction from the controller 15, the pairing connection unit 117 establishes a pairing connection with the selected device in accordance with a predetermined procedure.

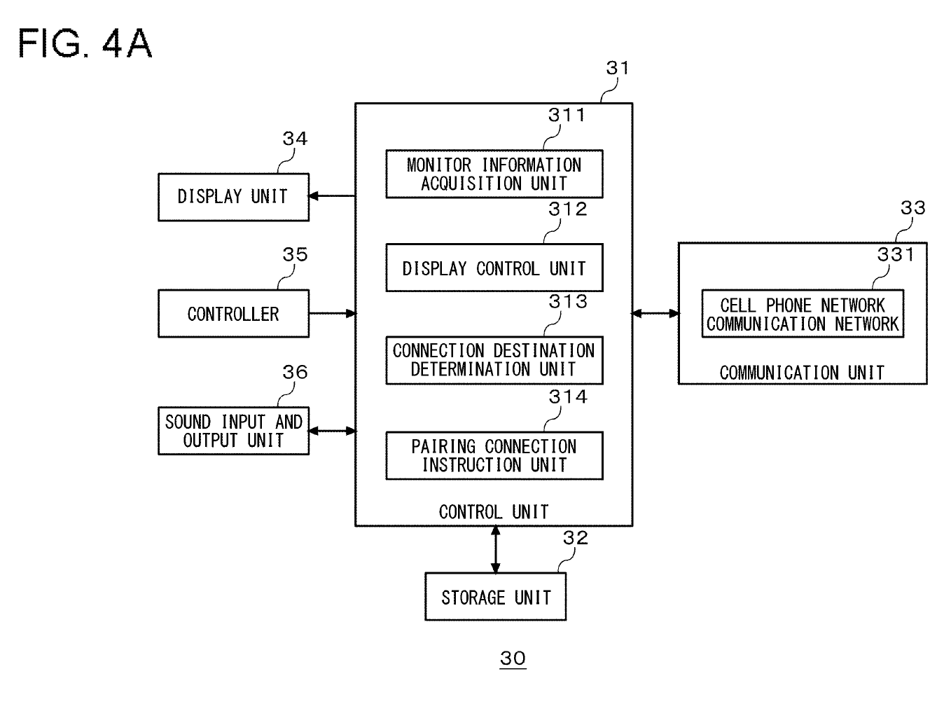

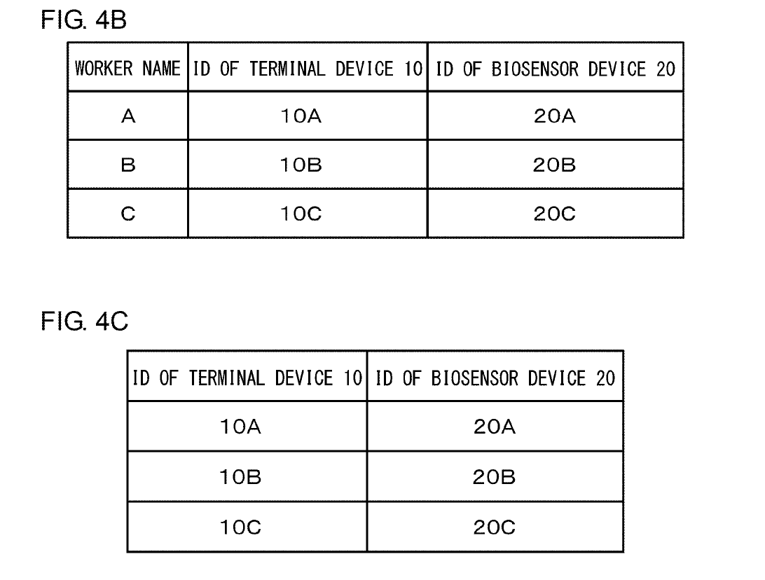

FIG. 4A shows the configuration of the management device 30 according to Embodiment 1. The management device 30 includes a control unit 31, a storage unit 32, a communication unit 33, a display unit 34, a controller 35, and a sound input and output unit 36. The control unit 31 includes a monitor information acquisition unit 311, a display control unit 312, a connection destination determination unit 313, and a pairing connection instruction unit 314. The functions of the control unit 31 are also implemented by the coordination of hardware resources and software resources, or hardware resources alone. FIG. 4 also depicts only those function blocks of the control unit 31 related to the process of interest in Embodiment 1.

The storage unit 32 includes a storage medium such as a nonvolatile semiconductor memory. As shown in FIG. 4B, the storage unit 32 stores, as device information, the name of a worker, the ID of the terminal device 10 carried by the worker, and the ID of the biosensor device 20 worn by the worker, mapping the information to each other. Further, as shown in FIG. 4C, the storage unit 32 keeps track of the conditions of pairing connection between the terminal device 10 and the biosensor device 20.

Before starting a firefighting activity, the worker establishes a pairing connection between the terminal device 10 carried by the worker and the biosensor device 20 worn by the worker. The storage unit 32 maintains pairing information as shown in FIG. 4C. The pairing information in the storage unit 32 is updated as appropriate when the monitor information is acquired or when a pairing disconnection instruction or a pairing connection instruction described below is communicated, as described below.

The sound input and output unit 36 includes a microphone and a speaker.

The monitor information acquisition unit 311 acquires the monitor information transmitted from the terminal device 10 via the cell phone network. The display control unit 312 displays the biological information included in the monitor information on the display unit 34, mapping the biological information to the name of the worker.

When the connection destination determination unit 313 acquires a trigger signal from the terminal device 10, the connection destination determination unit 313 determines a new destination of pairing connection (one of the other terminal devices 10) with the biosensor device 20 currently paired with the terminal device 10. A specific example of a method of determining a new destination of pairing connection will be described later.

The pairing connection instruction unit 314 provides the terminal device 10 currently paired with a given biosensor device 20 with a disconnection instruction to cancel the pairing connection with the biosensor device 20. Further, the pairing connection instruction unit 314 provides the terminal device 10 determined to be the new destination of pairing connection by the connection destination determination unit 313 with a pairing connection instruction to establish a pairing connection with the biosensor device 20. The pairing connection instruction includes the ID of the biosensor device 20 to identify the biosensor device 20 with which a pairing connection is established.

A description will now be given of the process performed during a firefighting operation at a scene of a disaster. As shown in FIG. 1, the biosensor devices 20a-20c are worn by the bodies of workers A-C. The workers A-C hold the terminal devices 10a-10c, respectively. The biosensor devices 20a-20c and the terminal devices 10a-10c are respectively paired by near field communication. The terminal devices 10a-10c are master modules and the biosensor devices 20a-20c are slave modules.

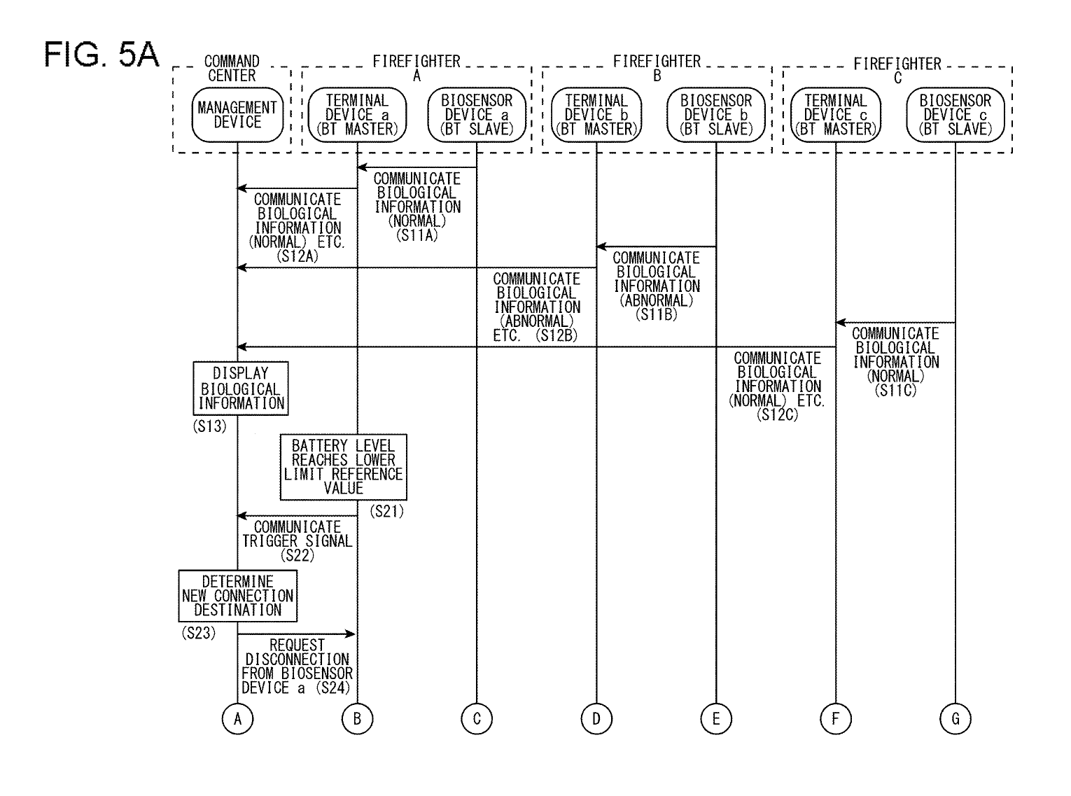

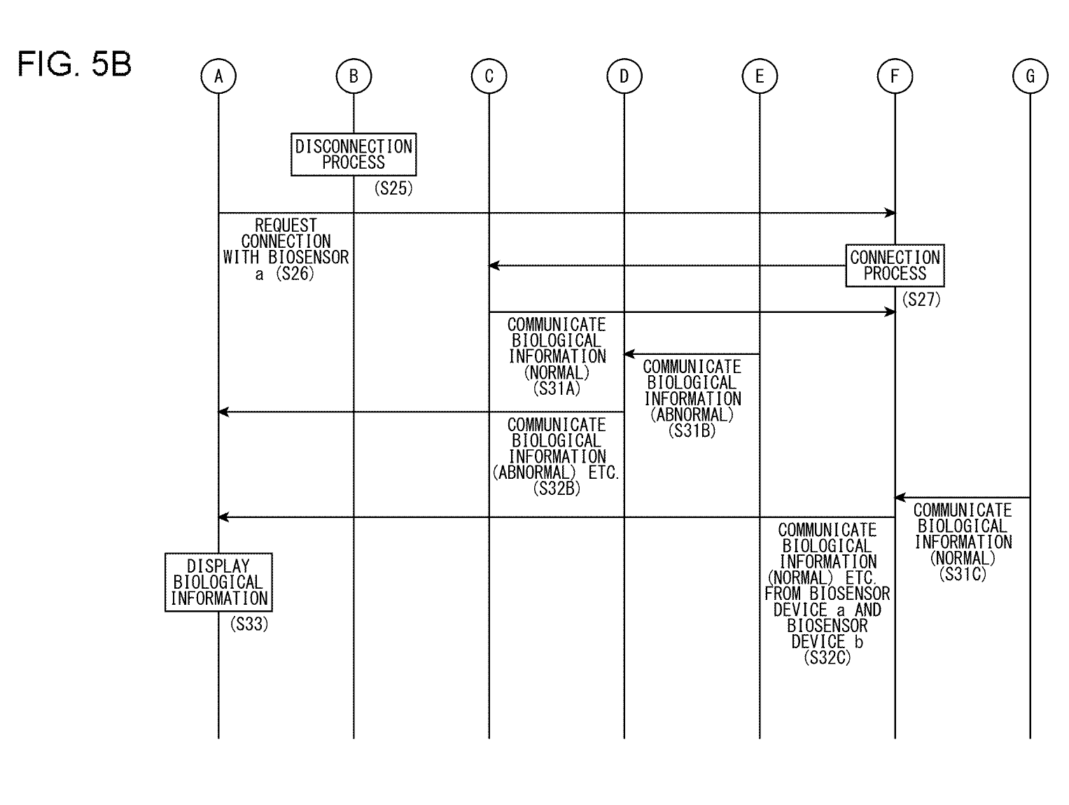

FIGS. 5A and 5B is a flowchart showing the flow of the process in the dangerous operation support system 1 according to Embodiment 1. The biological information acquisition unit 211 of the biosensor device 20a acquires biological information on worker A from the sensor unit 24. The biological information communicating unit 212 communicates the acquired biological information to the terminal device 10a by near field communication (S11A).

The biological information acquisition unit 111 of the terminal device 10a paired with the biosensor device 20a acquires the biological information and the ID of worker A from the biosensor device 20a by near field communication. The monitor information communicating unit 115 communicates monitor information including the biological information and the ID of worker A acquired by the biological information acquisition unit 111, the battery level, the position information, and the radio field intensity to the management device 30 via the cell phone network (S12A). The monitor information including the biological information on workers B and C is similarly communicated to the management device 30 of the command center 3 (S11B, S12B, S11C, S12C).

The monitor information acquisition unit 311 of the management device 30 acquires the monitor information including the biological information on workers A-C via the cell phone network. The display control unit 312 causes the display unit 34 to display the biological information on workers A-C included in the acquired monitor information (S13). The commander at the command center 3 monitors the biological information on workers A-C displayed on the display unit 34 and know the states of workers A-C.

A consideration is given of a situation in which the battery level of the terminal device 10a reaches a lower limit reference value (S21) and the battery level of the terminal device 10a will be depleted in a predetermined period of time (after several minutes). The trigger communicating unit 116 of the terminal device 10a provides the management device 30 with a trigger signal to indicate that the function of the terminal device 10a as a relay device (device responsible for relaying biological information) for the biosensor device 20a will not be fulfilled in a predetermined period of time (S22).

The trigger signal is also communicated to the biosensor device 20a (not shown). When the pairing connection unit 213 of the biosensor device 20a acquires the trigger signal, the pairing connection unit 213 discloses the existence of the biosensor device 20a (ID) to nearby devices provided with near field wireless communication functions. If the biosensor device is configured by default to return a response to search from other devices using near field communication, the process is not necessary.

When the connection destination determination unit 313 of the management device 30 acquires the trigger signal, the connection destination determination unit 313 determines the terminal device 10 that should be a new relay device for the biosensor device 20a (S23). In this process, the connection destination determination unit 313 examines the other terminal devices 10b and 10c and determines the terminal device 10 that should be a new destination of pairing connection, in accordance with the content of the monitor information relayed by the terminal devices 10b and 10c. More specifically, of the other terminal devices 10b and 10c, the connection destination determination unit 313 excludes the terminal device 10b relaying the biological information containing an abnormal detection value from the candidates of new pairing connection destination. The biological information containing an abnormal detection value is exemplified by biological information containing a detection value of the heart rate lower than a predetermined value causing one to estimate that the worker is in a dangerous condition. In the example shown in FIGS. 5A and 5B, the terminal device 10b relays the biological information containing an abnormal detection value. The terminal device 10c is relaying the biological information containing detection values in normal zones. Accordingly, the terminal device 10b is excluded from the candidates of new connection destination. Based on the foregoing, the connection destination determination unit 313 determines the terminal device 10c as a new destination of pairing connection with the biosensor device 20a.

The pairing connection instruction unit 314 of the management device 30 provides the terminal device 10a with a disconnection instruction directing the terminal device 10a to cancel the pairing connection with the biosensor device 20a (S24). When the pairing connection unit 117 of the terminal device 10a acquires the disconnection instruction, the pairing connection unit 117 performs a disconnection process to cancel the pairing connection with the biosensor device 20a (S25).

The pairing connection instruction unit 314 of the management device 30 provides the terminal device 10c with a connection instruction to establish a pairing connection with the biosensor device 20a (S26). When the pairing connection unit 117 of the terminal device 10c acquires the connection instruction, the pairing connection unit 117 performs a connection process of establishing a pairing connection with the biosensor device 20a (S27).

The biological information communicating unit 212 of the biosensor device 20a communicates the biological information on worker A to the newly paired terminal device 10c by near field communication (S31A). The biological information communicating unit 212 of the biosensor device 20b communicates the biological information on worker B to the terminal device 10b by near field communication (S31B). The biological information acquisition unit 111 of the terminal device 10b paired with the biosensor device 20b acquires the biological information on worker B from the biosensor device 20b by near field communication. The monitor information communicating unit 115 of the terminal device 10b communicates monitor information including the ID of the terminal device 10b, the biological information on worker B acquired by the biological information acquisition unit 111, the battery level, the position information, and the radio field intensity to the management device 30 via the cell phone network (S32B).

The biological information communicating unit 212 of the biosensor device 20c communicates the biological information on worker C to the terminal device 10c by near field communication (S31C). The biological information acquisition unit 111 of the terminal device 10c paired with both the biosensor devices 20a and 20c acquires the biological information on worker A from the biosensor device 20a and acquires the biological information on worker C from the biosensor device 20c by near field communication. The monitor information communicating unit 115 of the terminal device 10c communicates monitor information including the ID of the terminal device 10c, the biological information on worker A/the ID of the biosensor device 20a and the biological information on worker C/the ID of the biosensor device 20c acquired by the biological information acquisition unit 111, the battery level, the position information, and the radio field intensity to the management device 30 via the cell phone network (S32C).

The monitor information acquisition unit 311 of the management device 30 acquires the monitor information including the biological information on workers A-C via the cell phone network. The display control unit 312 causes the display unit 34 to display the biological information on workers A-C included in the acquired monitor information (S33).

In the example shown in the flowchart of FIGS. 5A and 5B, the terminal device 10c is uniquely determined to be a new destination of pairing connection, using a criterion requiring that the terminal device 10 relaying the biological information containing an abnormal detection value is excluded from the terminal devices 10 that are candidates of new pairing connection. The criterion alone may not be sufficient to uniquely determine a new destination of pairing connection. In this case, an additional criterion is necessary.

A consideration is given of a case where there are a plurality of terminal devices 10 relaying the biological information containing an abnormal detection value. In one exemplary approach, the connection destination determination unit 313 examines the plurality of terminal devices 10 relaying the biological information containing normal detection values and determines the terminal device 10 with the highest battery level as a new destination of pairing connection. In another exemplary approach, the connection destination determination unit 313 examines the plurality of terminal devices 10 relaying the biological information containing normal detection values and determines the terminal device 10 nearest the biosensor device 20a as a new destination of pairing connection. In still another exemplary approach, the connection destination determination unit 313 examines the plurality of terminal devices 10 relaying the biological information containing normal detection values and determines the terminal device 10 with the highest radio field intensity as a new destination of pairing connection.

An order of priority may be defined between the criterion using the battery level, the criterion using the distance to the target biosensor device 20, and the criterion using the radio field intensity. For example, given that the criteria are in the stated order of descending priority, the connection destination determination unit 313 determines the terminal device 10 closest to the target biosensor device 20 as a new destination of pairing connection, if the battery level of all of the plurality of terminal devices 10 relaying the biological information containing normal detection values is higher than a safety criterion (e.g., 80%).

A consideration will now be given of a case where there are no terminal devices 10 relaying the biological information containing normal detection values. In one exemplary approach, the connection destination determination unit 313 examines the terminal devices 10 other than the currently paired terminal device 10 and determines the terminal device 10 with the highest battery level as a new destination of pairing connection. In another exemplary approach, the connection destination determination unit 313 examines the terminal devices 10 other than the currently paired terminal device 10 and determines the terminal device 10 closest to the biosensor device 20a as a new destination of pairing connection. In still another exemplary approach, the connection destination determination unit 313 examines the terminal devices 10 other than the currently paired terminal device 10 and determines the terminal device 10 relaying the biological information showing the least serious abnormality as a new destination of pairing connection.

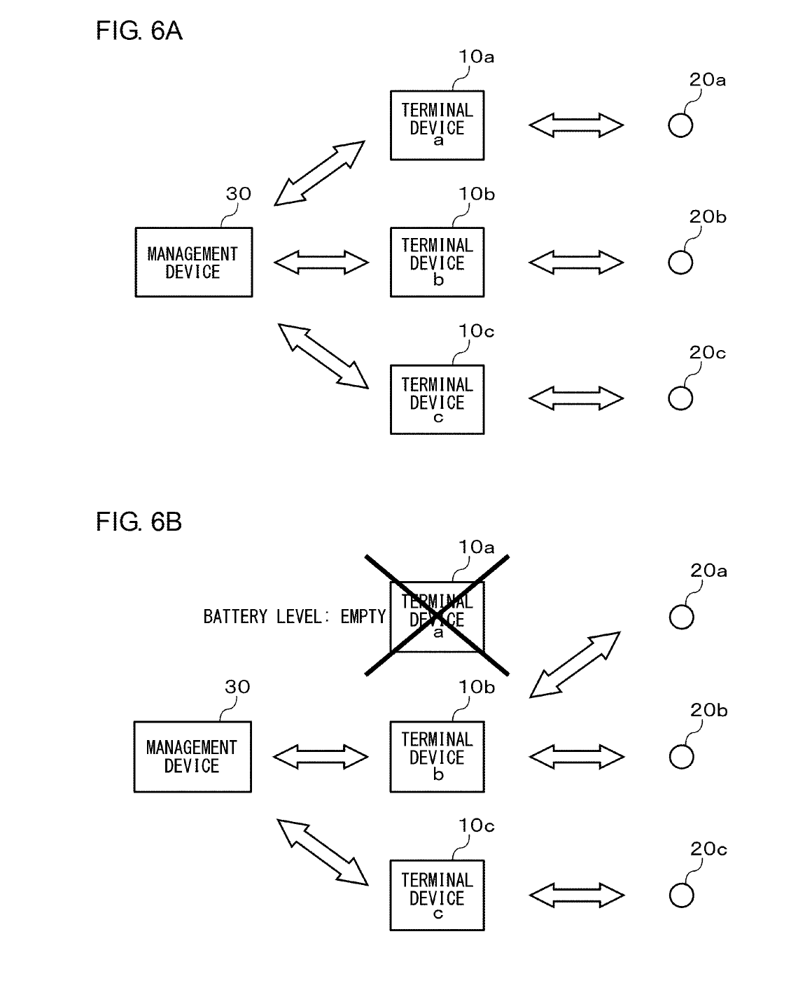

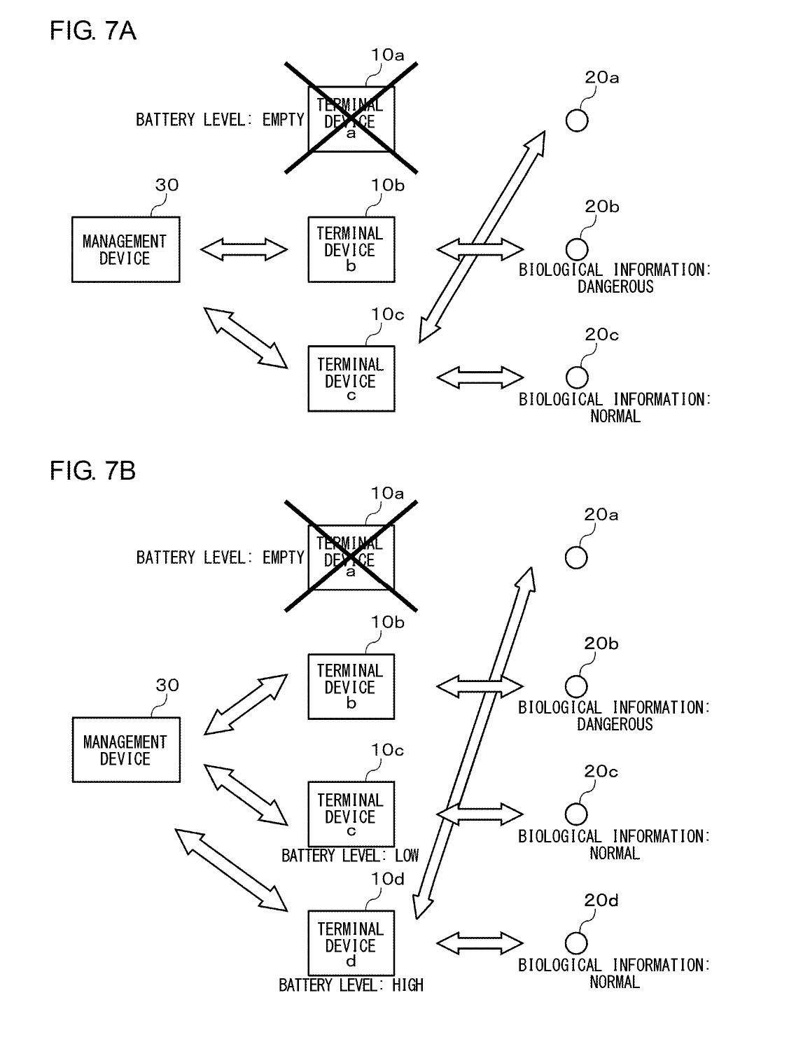

FIGS. 6A and 6B show specific examples of switching destinations of pairing connection according to Embodiment 1 (Case 1). FIGS. 7A and 7B show specific examples of switching destinations of pairing connection according to Embodiment 1 (Case 2).

FIG. 6A shows a state in which the battery level of the terminal devices 10a-10c held by workers A-C, respectively, is sufficient. The terminal devices 10a-10c are relaying the biological information on workers A-C to the management device 30 via communication channels that are respectively established.

FIG. 6B shows a state in which the battery level of the terminal device 10a held by worker A is depleted and the battery level of the terminal devices 10b and 10c held by workers B and C is sufficient. The distance from the biosensor device 20a worn by worker A to the terminal device 10b held by worker B is smaller than the distance to the terminal device 10c held by worker C. Therefore, the biosensor device 20a establishes a pairing connection with the terminal device 10b as a new destination of pairing connection. In addition to the biological information on worker B, the terminal device 10b relays the biological information on worker A to the management device 30.

FIG. 7A shows a state in which the battery level of the terminal device 10a held by worker A is depleted and the battery level of the terminal devices 10b and 10c held by workers B and C is sufficient. Further, the numerical value of the biological information on worker B relayed by the terminal device 10b to the management device 30 is in a dangerous zone and the numerical value of the biological information on worker C relayed by the terminal device 10c to the management device 30 is in a normal zone.

The distance from the biosensor device 20a worn by worker A to the terminal device 10b held by worker B is smaller than the distance to the terminal device 10c held by worker C. However, the numerical value of the biological information on worker B is in a dangerous zone so that the load on the terminal device 10b relaying the biological information on worker B should not be increased. Accordingly, the biosensor device 20a establishes a pairing connection with the terminal device 10c as a new destination of pairing connection. In addition to the biological information on worker C, the terminal device 10c relays the biological information on worker A to the management device 30.

FIG. 7B shows an example with four worker A-D. The battery level of the terminal device 10a held by worker A is depleted, the battery level of the terminal device 10c held by worker C is low, and the battery level of the terminal devices 10b and 10d held by workers B and D is sufficient. Further, the numerical value of the biological information on worker B relayed by the terminal device 10b to the management device 30 is in a dangerous zone, and the numerical values of the biological information on workers C and D relayed by the terminal devices 10c and 10d to the management device 30 are in a normal zone.

The distance from the biosensor device 20a worn by worker A to the terminal device 10b held by worker B is smallest, but the numerical value of the biological information on worker B is in a dangerous zone so that the load on the terminal device 10b relaying the biological information on worker B should not be increased. The distance from the biosensor device 20a worn by worker A to the terminal device 10c held by worker C is next smallest, but the battery level of the terminal device 10c is low so that the load on the terminal device 10c should not be increased. Accordingly, the biosensor device 20a establishes a pairing connection with the terminal device 10d as a new destination of pairing connection. In addition to the biological information on worker D, the terminal device 10d relays the biological information on worker A to the management device 30.

As described above, according to Embodiment 1, the relay device is switched from the currently paired terminal device 10 to the terminal device 10 held by another worker, if it is predicted to be difficult to communicate the biological information detected by the biosensor device 20 paired with the currently paired terminal device 10 to the management device 30 due to a low battery level of the currently paired terminal device 10, degradation in radio wave conditions, etc. In this way, a system can be built in which the biological information on workers constituting a team is communicated to the management device 30 more reliably.

In further accordance with Embodiment 1, the device responsible for relaying the biological information is switched from the currently paired terminal device 10 to another terminal device 10 by allowing for the content of biological information currently relayed by the candidate terminal device 10. To be more specific, the terminal device 10 relaying the biological information containing an abnormal detection value is excluded from the candidates of new pairing connection destination. This can prevent an increase in the load on the terminal device 10 relaying the biological information containing an abnormal detection value and prevents the battery level of the terminal device 10 from being reduced more rapidly. The communication channel for the biological information containing an abnormal detection value should continue to be reserved in preference to that of the biological information containing normal detection values. This is because the commander in command at the command center 3 often gives directions to rescue workers based on the biological information containing an abnormal detection value rather than based on the biological information containing normal detection values. In this respect, Embodiment 1 enables for preventing an increase in the load on the terminal device 10 relaying the biological information containing an abnormal detection value, thereby ensuring that the biological information on a worker in a dangerous condition is communicated to the management device 30 more reliably.

Further, a new destination of pairing connection is determined more properly by considering at least one of the battery level, the distance to the target biosensor device, and the radio field intensity, in addition to the content of biological information relayed.

### Embodiment 2

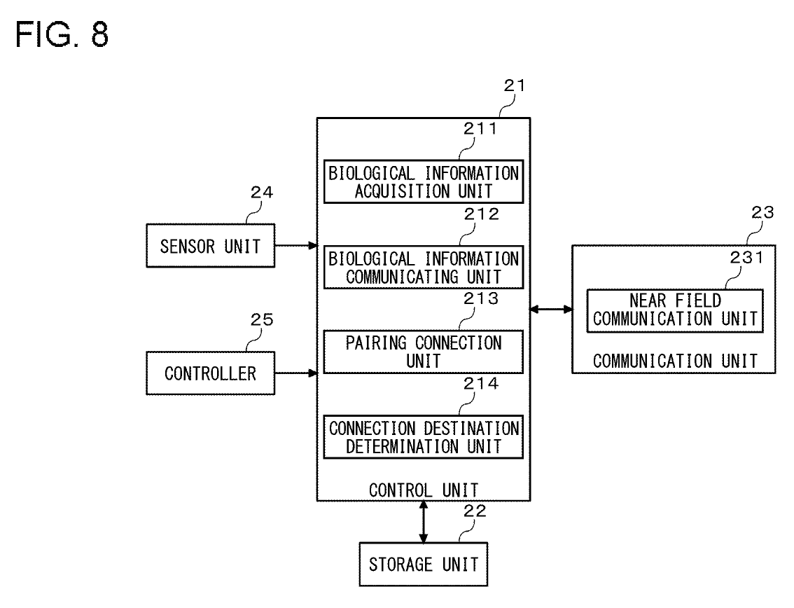

FIG. 8 shows the configuration of the biosensor device 20 according to Embodiment 2. The biosensor device 20 includes a control unit 21, a storage unit 22, a communication unit 23, a sensor unit 24, and a controller 25. The control unit 21 includes a biological information acquisition unit 211, a biological information communicating unit 212, a pairing connection unit 213, and a connection destination determination unit 214. In other words, the connection destination determination unit 214 is added to the configuration of the biosensor device 20 according to Embodiment 1 shown in FIG. 2.

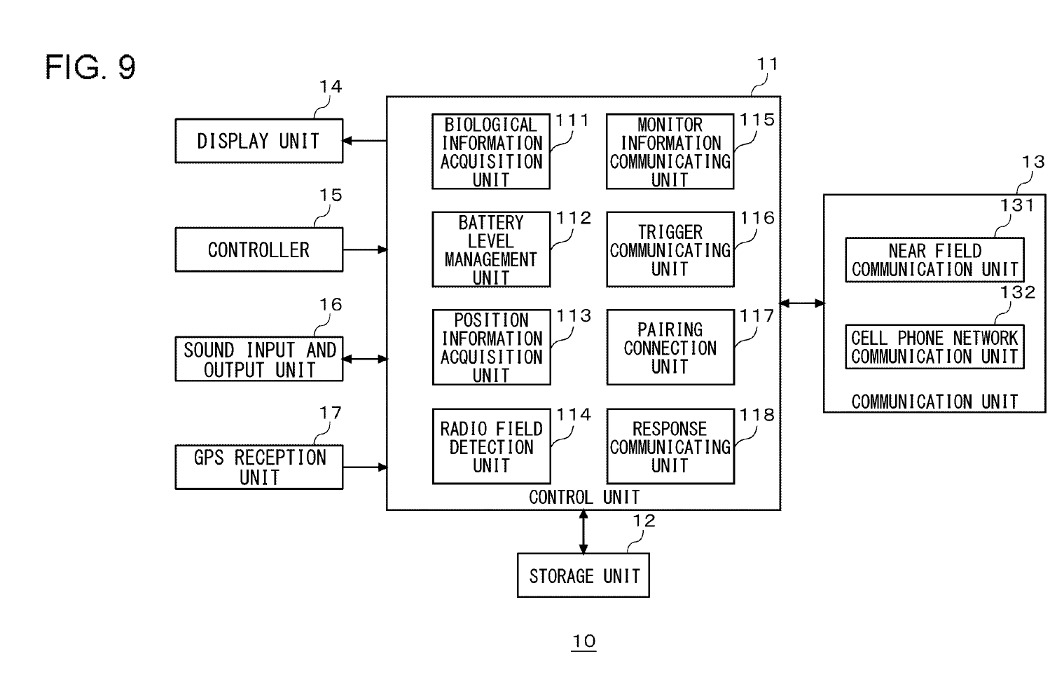

FIG. 9 shows the configuration of the terminal device 10 according to Embodiment 2. The terminal device 10 includes a control unit 11, a storage unit 12, a communication unit 13, a display unit 14, a controller 15, a sound input and output unit 16, and a GPS reception unit 17. The control unit 11 includes a biological information acquisition unit 111, a battery level management unit 112, a position information acquisition unit 113, an electric field detection unit 114, a monitor information communicating unit 115, a trigger communicating unit 116 a pairing connection unit 117, and a response communicating unit 118. In other words, the response communicating unit 118 is added to the configuration of the terminal device 10 according to Embodiment 1 shown in FIG. 3.

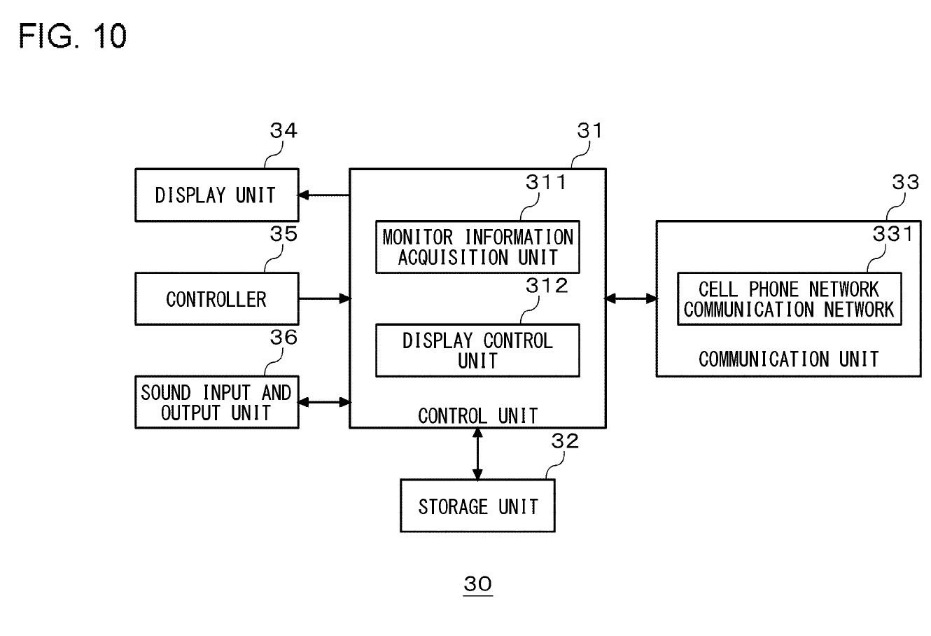

FIG. 10 shows the configuration of the management device 30 according to Embodiment 2. The management device 30 includes a control unit 31, a storage unit 32, a communication unit 33, a display unit 34, a controller 35, and a sound input and output unit 36. The control unit 31 includes a monitor information acquisition unit 311 and a display control unit 312. In other words, the connection destination determination unit 313 and the pairing connection instruction unit 314 are removed from the configuration of the management device 30 according to Embodiment 1 shown in FIG. 1.

Thus, Embodiment 2 is configured to transfer the connection destination determination process of the management device 30 according to Embodiment 1 to the biosensor device 20a.

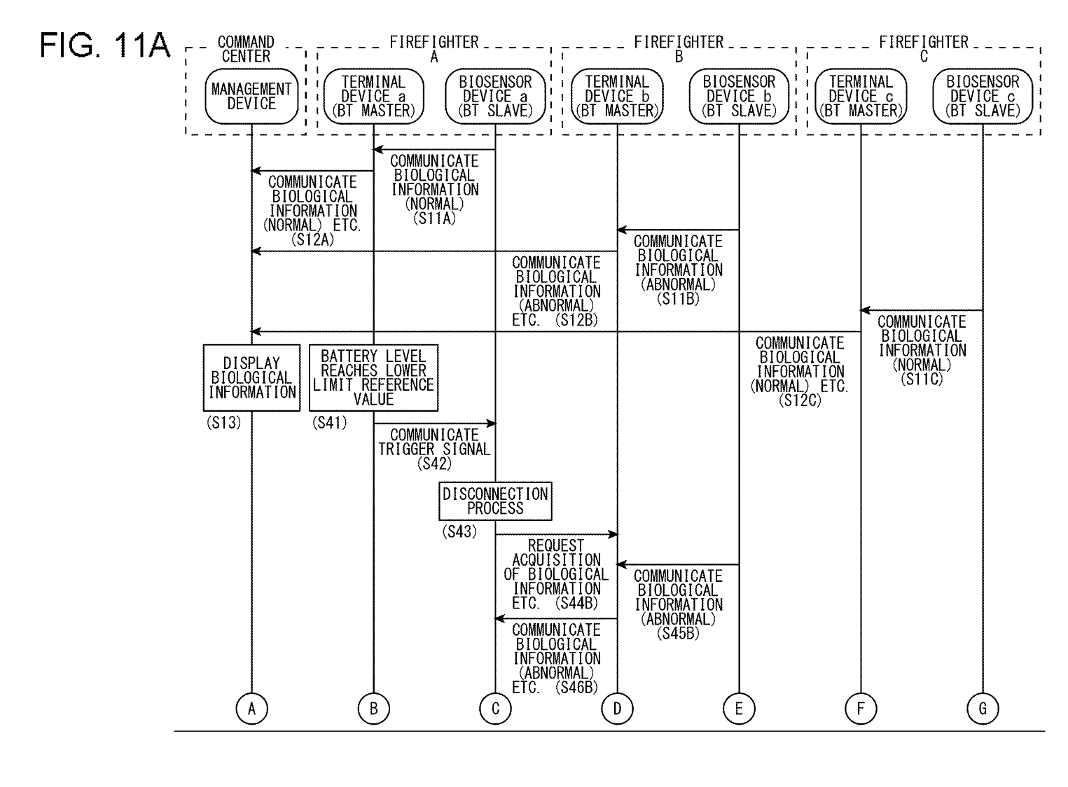

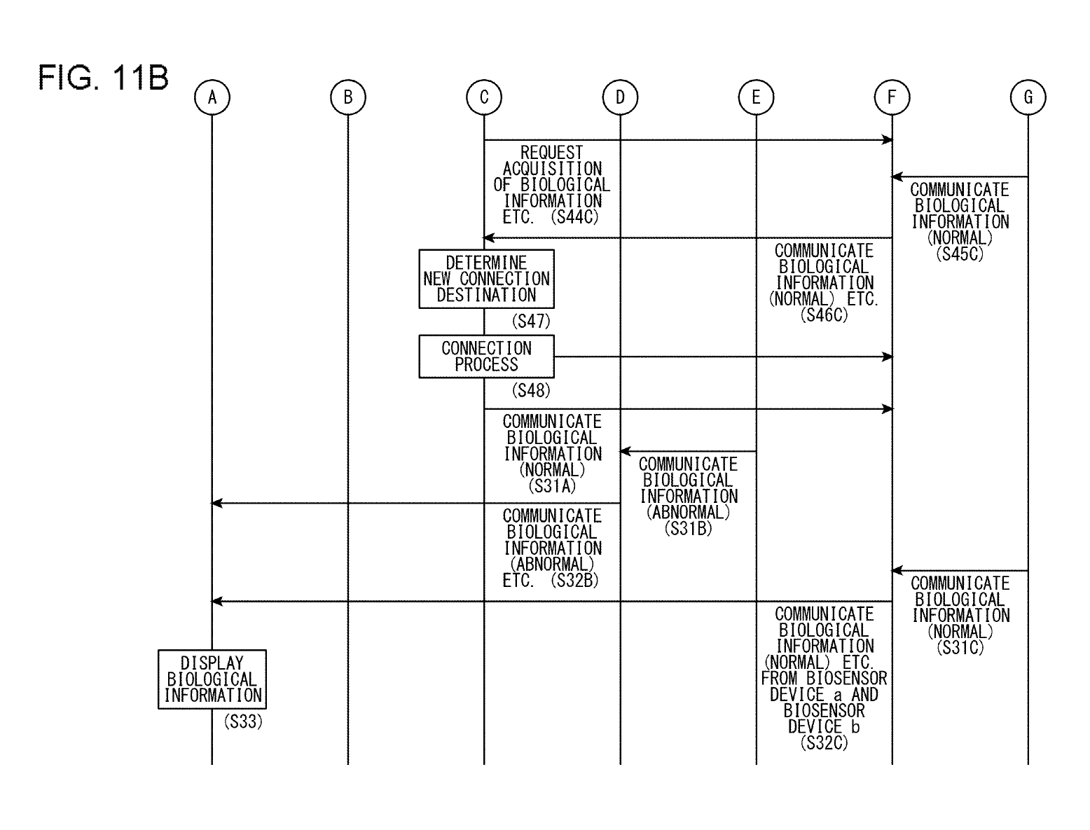

FIGS. 11A and 11B is a flowchart showing the flow of the process in the dangerous operation support system 1 according to Embodiment 2. Steps S11-S13 are similar to steps S11-S13 of the flowchart of FIGS. 5A and 5B according to Embodiment 1.

A consideration is given of a situation in which the battery level of the terminal device 10a reaches a lower limit reference value (S41) and the battery level of the terminal device 10a will be depleted in a predetermined period of time (after several minutes). The trigger communicating unit 116 of the terminal device 10a provides the biosensor device 20a of a trigger signal to indicate that the function of the biosensor device 20a as a relay device for the biosensor device 20a will not be fulfilled in a predetermined period of time (S42).

When the pairing connection unit 213 of the biosensor device 20a acquires the trigger signal, the pairing connection unit 213 performs a disconnection process to cancel the pairing connection with the terminal device 10a (S43). The connection destination determination unit 214 causes the near field communication unit 231 to transmit a search signal including a request for transmission of a response signal containing the biological information relayed (S44B, S44C). The terminal device 10 located in a range reached by radio waves of near field communication from the biosensor device 20a receives the search signal. In the example shown in FIGS. 11A and 11B, both the terminal device 10b and the terminal device 10c receive the search signal.

The biological information acquisition unit 211 of the biosensor device 20b acquires biological information on worker B from the sensor unit 24. The biological information communicating unit 212 communicates the acquired biological information to the terminal device 10b by near field communication (S45B). The response communicating unit 118 of the terminal device 10b causes the near field communication unit 131 to transmit a response signal containing the ID of the terminal device 10b, the biological information on worker B, and the ID of the biosensor device 20b, the battery level, and the position information (S46B).

Similarly, the biological information acquisition unit 211 of the biosensor device 20c acquires biological information on worker C from the sensor unit 24. The biological information communicating unit 212 communicates the acquired biological information to the terminal device 10c by near field communication (S45C). The response communicating unit 118 of the terminal device 10c causes the near field communication unit 131 to transmit a response signal containing the ID of the terminal device 10c, the biological information on worker C, and the ID of the biosensor device 20c, the battery level, and the position information (S46C).

When the connection destination determination unit 214 of the biosensor device 20a acquires the response signal from the terminal devices 10b and 10c, the connection destination determination unit 214 determines the terminal device 10 that should be a new relay device for the biosensor device 20a (S47). In this process, the connection destination determination unit 214 examines the terminal devices 10b and 10c and determines the terminal device 10 that should be a new destination of pairing connection in accordance with the content of the monitor information relayed by the terminal devices 10b and 10c. More specifically, of the terminal devices 10b and 10c, the connection destination determination unit 214 excludes the terminal device 10b relaying the biological information containing an abnormal detection value from the candidates of new pairing connection destination. In the example shown in FIGS. 11A and 11B, the terminal device 10b relays the biological information containing a detection value in a dangerous zone. The terminal device 10c is relaying the biological information containing detection values in normal zones. Accordingly, the terminal device 10b is excluded from the candidates of new connection destination. Based on the foregoing, the connection destination determination unit 214 determines the terminal device 10c as a new destination of pairing connection with the biosensor device 20a.

The pairing connection unit 213 of the biosensor device 20a performs a connection process of establishing a pairing connection with the terminal device 10c (S48). The steps subsequent to the establishment of pairing connection between the biosensor device 20a and the terminal device 10c are similar to steps S31-S33 of the flowchart of FIGS. 5A and 5B according to Embodiment 1.

In addition to the criterion requiring that the terminal device 10 relaying the biological information containing an abnormal detection value be excluded, the connection destination determination unit 214 of the biosensor device 20a may use an additional criterion for determination. The additional criterion for determination may be the additional criterion for determination described in Embodiment 1.

As described above, Embodiment 2 provides the same advantage as Embodiment 1. In further accordance with Embodiment 2, only the terminal device 10 returning a response signal in response to a search signal transmitted from the biosensor device 20 using near field communication can be a candidate of new pairing connection destination. This ensures that the terminal device 10 that can be paired reliably by near field communication can be defined as a candidate of destination of connection.

Described above is an explanation based on an exemplary embodiment. The embodiment is intended to be illustrative only and it will be obvious to those skilled in the art that various modifications to constituting elements and processes could be developed and that such modifications are also within the scope of the present invention.

In the embodiments described above, the monitor information communicated from the terminal device 10 to the management device 30 includes the battery level, the position information, and the radio field intensity of the terminal device 10. These items of information need not necessarily be included in the monitor information and can be omitted as necessary. For example, if the terminal device 10 is of a type not provided with the GPS reception unit 17, the position information is omitted.

If the position information is omitted, the biosensor device 20a or the terminal device 10a transmits a search signal using near field communication and receives a response signal from the terminal device 10 receiving the search signal. By detecting the radio field intensity of the response signal, the biosensor device 20a or the terminal device 10a can estimate the distance between the biosensor device 20a or the terminal device 10a and the terminal device 10 held by another worker. If the battery level is of no concern, the biosensor device 20a establishes a pairing connection with the most closely located terminal device 10 held by another worker.

In the embodiments described above, a trigger signal is communicated to the management device 30 or the biosensor device 20a when the battery level of the terminal device 10a reaches a lower limit reference value, and the management device 30 or the biosensor device 20a is caused to determine a new destination of pairing connection. Alternatively, the terminal device 10a may itself perform the process described in Embodiment 1 or Embodiment 2 so as to determine a new destination of pairing connection.

What is claimed is:

1. A biosensor device comprising:
a biological information acquisition unit that acquires biological information on a subject person;
a communication unit that transmits the biological information acquired by the biological information acquisition unit to a communication terminal device that serves as a relay device for relaying the biological information to a management device; and
a connection destination determination unit that, when changing a destination of connection from a currently connected communication terminal device to another communication terminal device, determines a communication terminal device as a new destination of connection in accordance with a content of the biological information relayed by the other communication terminal devices.

2. The biosensor device according to claim 1, wherein
the connection destination determination unit causes the communication unit to transmit a search signal including a request for transmission of a response signal including the biological information relayed and to receive the response signal transmitted from the communication terminal device receiving the search signal.

3. The biosensor device according to claim 1, wherein
when the connection destination determination unit receives, from a currently connected communication terminal, a notification signal indicating that a battery level is lower than a preset value or a notification signal indicating that a received radio field intensity is lower than a preset value, the connection destination determination unit starts a process of determining a communication terminal device as a new destination of connection.

4. The biosensor device according to claim 1, wherein
the connection destination determination unit excludes the communication terminal device relaying the biological information containing an abnormal detection value from candidates of new destination of connection.

5. The biosensor device according to claim 1, wherein
the connection destination determination unit causes the communication unit to transmit a search signal including a request for transmission of a response signal including the biological information relayed and a battery level and to receive the response signal transmitted from the communication terminal device receiving the search signal, and determines a communication terminal device as a new destination of connection in accordance with a content of the biological information relayed and the battery level.

6. A management device comprising:
a communication unit that receives a signal including biological information on a subject person detected by a biosensor device, via a communication terminal device; and
a connection destination determination unit that, when changing a relay device for a given biosensor device from a currently connected communication terminal device to another communication terminal device, determines a communication terminal device as a new destination of connection in accordance with a content of the biological information relayed by the other communication terminal devices.

7. The management device according to claim 6, wherein
the communication unit transmits a signal to the communication terminal device as a new destination of connection determined by the connection destination determination unit to direct the determined communication terminal device to perform a process of connecting to the biosensor device.

8. The management device according to claim 6, wherein
when the connection destination determination unit receives, from a currently connected communication terminal, a notification signal indicating that a battery level is lower than a preset value or a notification signal indicating that a received radio field intensity is lower than a preset value, the connection destination determination unit starts a process of determining a communication terminal device as a new destination of connection.

9. The management device according to claim 6, wherein
the connection destination determination unit examines said at least one other communication terminal devices and excludes the communication terminal device relaying the biological information containing an abnormal detection value from candidates of new destination of connection.

10. The management device according to claim 6, wherein
the communication unit receives, from the communication terminal device, a signal including the biological information and a battery level of the communication terminal device, and
the connection destination determination unit determines a communication terminal device as a new destination of connection in accordance with a content of the biological information and the battery level.

11. A communication system comprising:
a biosensor device that acquires biological information on a subject person;
a plurality of communication terminal devices that relay the biological information acquired by the biosensor device to a management device; and
a management device that acquires the biological information on a plurality of persons from the plurality of communication terminals, wherein
the management device:
when changing a relay device for a given biosensor device from a currently connected communication terminal device to another communication terminal device, determines a communication terminal device as a new destination of connection in accordance with a content of the biological information relayed by the other communication terminal devices.
